(19) United States
(12) Patent Application Publication
(10) Pub. No.: US 2026/0060807 A1
(43) Pub. Date: Mar. 5, 2026

(54) APPARATUS FOR COATING IMPLANTS AND ASSOCIATED METHOD

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 19/313,281

(22) Filed: Aug. 28, 2025

(30) Foreign Application Priority Data
Sep. 3, 2024 (EP) 24198143.0

(51) Int. Cl. A61F 2/30; A61L 27/54

(52) U.S. Cl. CPC A61F 2/30767; A61L 27/54; A61F 2310/0097; A61L 2420/02

(57) ABSTRACT

An apparatus for coating elongate implants, a system comprising such an apparatus, a use of an apparatus and a method for coating an elongate implant. An apparatus for coating an elongate implant comprises a reservoir for holding a coating agent and a lower part connected or connectable to the reservoir and having an opening. A material forming the opening is rubber-elastic and serves as a wiper for wiping off excess coating agent.

### CROSS-REFERENCE TO RELATED APPLICATION

This application claims priority pursuant to 35 U.S.C. 119(a) to European Patent Office Application No. 24198143.0, filed Sep. 3, 2024, which application is incorporated herein by reference in its entirety.

### FIELD

The invention relates to an apparatus for coating elongate implants, a system comprising such an apparatus, a use of an apparatus and a method for coating an elongate implant.

### BACKGROUND

Intramedullary nails and osteosynthesis plates are used to treat bone fractures, for example in trauma surgery. This occurs particularly in the case of fractures of long bones, such as the thigh bone (femur), shin bone (tibia) or upper arm bone (humerus). After such treatment, infections of the surrounding bone and/or soft tissue can occur, for example with bacteria, such as Staphylococcus aureus or Staphylococcus epidermidis. This necessitates surgical debridement, during which infected tissue is removed. In addition, systemic antibiotics and/or local anti-infective agents are used to combat the infection on a targeted basis.

It would be desirable if mechanical stabilization of the fractured bone tissue were still possible after surgical debridement, for example through elongate implants, such as intramedullary nails and/or osteosynthesis plates, and if these could simultaneously release local anti-infective agents in order to locally suppress microbial germs remaining in the debrided tissue.

It is known to use intramedullary nails with an antibiotic-containing coating of polymethyl methacrylate bone cement (N. Walter et al.: “Individual and commercially available antimicrobial coatings for intramedullary nails for the treatment of infected long bone non-unions—a systematic review”; Injury 2022; DOI: 10.1016/j.injury.2022.05.008).

Antibiotics from the bone cement are dissolved from the coating by aqueous body fluids, such as wound secretions and blood, leading to locally high antibiotic concentrations. In combination with systemic antibiotics, the germs that may remain after debridement can be reduced.

It is also desirable to produce such a coating intraoperatively. This makes it possible to incorporate into the coating agent anti-infective agents that are precisely tailored to the germs present. To date, intraoperative coating has been carried out by preparing bone cement with the required anti-infective agents and manually forming a layer of bone cement around the intramedullary nail to be coated. It is very difficult to achieve a uniform layer thickness over the entire length of the intramedullary nail. It often happens that the coated nail comprises at least a partially too large cross-section and can no longer be implanted into the debrided bone.

Due to the strong adhesion of polymethyl methacrylate bone cement to metal surfaces, the applied bone cement can no longer be removed from the surface of the intramedullary nail after a short period of time. In some regions, layer thickness is also too low, so that at least locally, inadequate amounts of anti-infective agents are available.

U.S. Patent Publication No. 2007/0134287A1 describes an antibiotic coating solution that consists of a readily evaporating solvent and an antibiotic dissolved therein. U.S. Pat. No. 11,517,650 describes a similar coating solution, in which antibiotics are dissolved in a rapidly or moderately evaporating solvent and energy is supplied by ultrasound to accelerate the dissolution process. Printed publication EP1243259B1 discloses a coating that consists of polymethyl methacrylate and a hydrophilic polymer, in which an antibiotic is suspended. A similar solution is described in EP1112095B1. Here, a suspension is applied to implant surfaces, the solvent evaporates and a D,L-polylactide film remains in which antibiotic particles are fixed.

### SUMMARY

The above-mentioned features can be combined as desired with the different aspects of the invention.

The object of the invention is to provide elongate implants having a defined coating in a simple manner.

The object is achieved by the apparatus according to claim 1 and by the system, the use and the method according to the additional independent claims. Advantageous embodiments are specified in the dependent claims.

For achieving the object, an apparatus for coating an elongate implant is used. The apparatus comprises a reservoir for holding a coating agent along with a lower part. In particular, the lower part is connected or connectable to the reservoir. The lower part comprises an opening. The material forming the opening is rubber-elastic and serves as a wiper for wiping off excess coating agent.

During intended use, the reservoir is located above the lower part. A coating agent is located in the reservoir. An elongate implant is moved downward through the reservoir and opening, typically along the central axis of the apparatus. The implant can be wetted with the coating agent in the reservoir and/or in underlying regions of the apparatus. The wiper removes excess coating agent from the surface of the implant. In this way, a uniform coating is made possible. The region forming the opening adapts to the shape of the implant and thus makes uniform wiping possible. The coating can subsequently cure within minutes.

The apparatus is simple and cost-effective to produce, easy to use and allows the reproducible production of defined coatings. Due to the simple and rapid application, this can also be carried out intraoperatively, so that a coating having an individually adapted active ingredient can be used.

The coating agent can comprise, for example, bone cement or polymer. Alternatively, or additionally, the coating agent can comprise one or more active ingredients, such as, e.g., anti-infective agents. Active ingredients can be dissolved or suspended in one or more polymers. The implant can be, for example, an intramedullary nail or an osteosynthesis plate.

The reservoir is open, in particular, at the top or on the side facing away from the opening. In this way, the implant and/or coating agent can be inserted or added into the reservoir from above.

The reservoir can comprise a rotationally symmetrical basic shape. “Rotationally symmetrical basic shape” means that the underlying shape is rotationally symmetrical. There may be attachments, additional parts, recesses, holes, etc. on or in this basic shape, so that the actual shape may deviate from the rotationally symmetrical shape. There may also be other deviations from the rotationally symmetrical shape, such as, e.g., thickenings, flattenings, etc.

The reservoir encloses a cavity for holding the coating agent. The lower part is the region of the apparatus forming the opening. The opening serves for the exit of the implant from the apparatus after coating. The wall of the opening can be designed as a wiper. The wiper typically comprises an edge that serves to wipe and can also be referred to as a wiper lip. The wiper is designed in such a way that a viscous liquid cannot flow between the wiper and the implant.

The lower part is mechanically connected or connectable to the reservoir. A manual and/or reversible connection can be provided. The connection is particularly liquid-tight. The lower part can be indirectly connected or connectable to the reservoir.

The material forming the opening is made of a rubber-elastic material. In particular, the lower part is at least partially made of rubber-elastic material. This makes elastic deformation of the opening possible. Typically, the rubber elasticity is such that a body having a diameter 10% larger than the diameter of the opening can be passed through the opening without being damaged. This also applies preferably to diameters that are 20% or 30% larger. In particular, the opening having the wiper is slightly smaller than the implant to be coated. Thus, an uncontrolled flow of the coating agent can be prevented.

The rubber elasticity also allows the coating of implants with a variable extension along the longitudinal axis, e.g., a variable diameter. Such implants are frequently used in practice. Due to stretching of the material forming the opening, a defined coating is made possible.

An elongate implant within the meaning of the invention is an implant whose extension in a main extension direction is greater by a factor of at least 3 than in all directions transverse to the main extension direction. A cross-section of the implant can, for example, be circular, elliptical or polygonal. Polygonal cross-sections have at least three corners. The implant can be rod-shaped.

In one embodiment, the lower part comprises a hollow conical portion. In particular, the conical portion is partially or completely made of rubber-elastic material.

The apparatus is therefore constructed in two parts. An upper part is defined by the reservoir, and the lower part comprises the conical portion. During intended use, the reservoir is typically located above the conical portion. A coating agent can be in the reservoir and/or in the conical portion. The implant can come into contact with the coating agent in the reservoir and/or in the conical portion. The conical portion can adapt to the shape of the implant, thus making a particularly effective coating possible.

The lower part can connect the reservoir to the opening and/or serve to coat the implant. The lower part and/or the conical portion typically define a cavity in which coating agent can be held and/or through which the implant can be moved. The cavity of the reservoir is connected to the cavity of the lower part or the conical portion. In particular, the opening is arranged on the side of the conical portion facing away from the reservoir.

The conical portion comprises in particular a conical outer surface. The inner surface can also be conical in shape. The wall thickness can be constant. The central axis of the conical portion extends from a larger first side to a smaller second side. The conical portion can comprise a rotationally symmetrical, rotationally symmetrical (about an axis), mirror-symmetrical and/or elongate basic shape. The central axis can coincide with the rotation axis, the symmetry axis and/or a central axis of a symmetry plane. The conical portion is particularly elongated in shape. The longitudinal axis of the conical portion can therefore coincide with the central axis. The conical portion can comprise the shape of a hollow truncated cone. The opening is located in particular on the smaller second side and/or the side of the conical portion facing away from the reservoir.

An angle between an outer side of the conical portion and the central axis of the conical portion is typically at most 45°, preferably at most 35° and in particular at most 25°. Particularly preferably, the angle is at most 20° and in particular at most 15°. The angle is typically at least 2°, in particular at least 5°.

Preferably, the lower part is mechanically connected or connectable to the reservoir. A manual and/or reversible connection can be provided. The connection is particularly liquid-tight.

In particular, the conical portion is made partly or entirely of a rubber-elastic material. This makes elastic deformation possible.

In one embodiment, the material forming the opening, a material of the lower part or a material of the conical portion is a material having a Shore A hardness of at least 30, preferably at least 50. In particular, the Shore A hardness of the material is at most 80.

In one embodiment, a larger first side of the conical portion points toward the reservoir and a smaller second side of the conical portion points away from the reservoir. In particular, the opening is located on the second side.

The conical portion comprises two sides, in particular at opposite positions in relation to a longitudinal axis and/or a central axis of the conical portion. The larger side is larger than the smaller side. The first side is connected or connectable to the reservoir. The wiper can be arranged on the second side. The wiper is located in particular at the end of the conical portion pointing away from the reservoir or free end.

The first side comprises an opening that is continuously connected to the reservoir. The extension of this opening, for example the free diameter, corresponds at least to the transverse extension, for example the outer diameter, of the implant to be coated.

In one embodiment, the reservoir comprises a first connecting region and the lower part comprises a corresponding second connecting region. In particular, the first connecting region and the second connecting region can be mechanically reversibly connected to one another.

The lower part and the upper part can therefore be connected reversibly. “Reversible” means that the connection can be released without causing damage. In particular, the connection can be established and/or released manually. For example, the connecting regions are configured as corresponding threads, as corresponding parts of a bayonet lock or as corresponding parts of a plug connection.

This makes possible the easy replacement of the conical portion or installation of a desired conical portion. Different conical portions can be provided for different implants. For example, a conical portion for an implant having a circular cross-section can comprise an opening having a circular basic shape and/or a conical portion for a flat implant can comprise an opening having an elongate basic shape. The actual shape may differ from the basic shape, e.g., due to spacers. On the side facing the reservoir, however, the different conical portions can be of identical or similar shape.

In one embodiment, the opening exhibits a circular or slit-shaped basic shape. In this way, intramedullary nails having a circular cross-section or osteosynthesis plates having a flat cross-section can be coated particularly uniformly. The actual shape of the opening may differ from the basic shape, for instance due to the spacers.

In one embodiment, spacers are arranged on the wiper in order to ensure a distance between the wiper and an elongate implant to be coated. Spacers ensure a defined distance between the wiper and the implant if the implant is moved through the opening. This makes possible a coating with a uniform layer thickness.

Since the spacers contact the implant directly, there is less or no coating agent at these contact points. Longitudinal strips without coating then form on the surface of the implant. It has been shown that with some coating agents, for example low-viscosity bone cements, the coating agent also flows into the non-coated regions after coating, in this way achieving a uniform coating.

In particular, the spacers protrude radially inwards. Preferably, the spacers are designed so that most of the surface of the implant is coated. The proportion of the surface of the implant to which the coating agent is applied is thus at least as large and preferably larger than the proportion of the surface that is in contact with the spacers. It has been shown that a coating of at least 50% of the surface is sufficient to ensure an effective supply of the active ingredient. Preferably, the spacers are arranged at equal distances to ensure uniform application of the coating.

The spacers are arranged in particular on an inner surface of the conical portion and/or the wiper. In this way, a distance between the wiper and the implant can be ensured when wiping off the coating agent.

In particular, the spacers are arranged in a manner distributed in the longitudinal direction, preferably at equal distances. The spacers can be designed to be, e.g., pin-shaped or rib-shaped.

The spacers are particularly fastened or molded onto the conical region. In particular, the spacers are not made of the rubber-elastic material of the conical portion, but of a material having a higher stiffness.

In the case of a slit-shaped opening, it can be provided that only the long sides viewed in cross-section are coated. There are then no spacers arranged on the narrow sides. Alternatively, or additionally, it can be provided to coat only one side. On the side not to be coated, no spacers are then arranged, so that the coating agent is completely or almost completely wiped off.

The spacers are typically not made of the rubber-elastic material forming the opening. Spacers are typically made of harder and/or stronger material. Rubber-elastic yielding of the spacers is not desired. In one embodiment, the spacers are made of plastic, preferably a thermoplastic.

The spacers can be fastened to the conical portion. The spacers can alternatively or additionally be fastened to a separate structure, in particular the lower part. In this case, it is possible that the spacers are in contact with the conical portion.

In one embodiment, the spacers are arranged in a manner uniformly distributed in the circumferential direction. This allows the coating to be applied uniformly. In particular, at least three spacers are present in the circumferential direction, preferably at least four or at least five spacers. In one embodiment, six or more spacers are arranged in the circumferential direction.

In one embodiment, the spacers are configured to be rib-shaped. “Rib-shaped” means a shape in which a first extension direction is many times longer than the second and third extension directions, both of which are perpendicular to the first and to one another. For example, the first extension direction may be greater than the second and/or third extension direction by a factor of at least 3, preferably at least 5 and particularly preferably at least 8 or 10. The rib-shaped spacers can be arranged on the conical portion.

Preferably, the rib-shaped spacers extend in the longitudinal direction of the apparatus and/or the conical portion.

In one embodiment, the spacers extend at an angle of less than 30° to a central axis of the apparatus. However, due to the conical shape of the conical portion, the spacers do not extend exactly parallel to the central axis or the outer wall of the conical portion, but, like the outer side of the conical portion, at an angle thereto. The angle between a longitudinal axis of a rib-shaped spacer and the central axis of the conical portion can in principle be at most 45°, preferably at most 35° and in particular at most 25°. Particularly preferably, the angle is at most 20° and in particular at most 15°. The angle is typically at least 2°, in particular at least 5°.

Due to their elongate shape, the spacers cannot yield longitudinally. This ensures that the distance is maintained in a particularly safe manner if the implant is moved along the central axis through the apparatus. The rib-shaped spacers allow the production of a coating having a constant thickness even in the event of stretching of the material forming the opening due to a changing cross-section of the implant.

In one embodiment, the rib-shaped spacers are shaped so that they touch a maximum of 35% of the circumference of the implant, preferably a maximum of 25% and particularly preferably a maximum of 15%.

In one embodiment, the rib-shaped spacers comprise a length of at least 3 mm, preferably at least 5 mm and/or at most 20 mm, preferably at most 15 mm, along their main extension direction. In one embodiment, the rib-shaped spacers comprise a width of at least 0.5 mm, in particular at least 1.0 mm and/or at most 5.0 mm, in particular at most 3.0 mm, in a direction transverse to their main extension direction. This can apply to both directions transverse to the main extension direction. The two directions can comprise the same or different extensions. The extension of the spacers in the radial direction defines the layer thickness. In particular, all spacers comprise the same extensions at least in the radial direction and preferably also in all other directions.

In one embodiment, the rib-shaped spacers comprise a cross-section having a contact side and a free side. In particular, a first width of the spacers on the contact side is at least in some regions larger than the second width of the spacers on the free side, measured parallel to the first width. The contact side rests against the conical portion and can be fastened thereto. The free side protrudes freely inwards and serves to contact the implant.

In other words, the spacers comprise a transverse extension measured perpendicular to the longitudinal extension, which is at least in some regions wider in the region of an inner wall of the conical portion than in the region of the free end.

In this way, the contact area between the implant and the spacer is minimized. As a result, the uncoated portion of the surface of the implant is reduced. In this way, a particularly uniform coating can be achieved.

In one embodiment, the rib-shaped spacers comprise a triangular, trapezoidal, rectangular or hemispherical cross-section. Such a shape of the spacer minimizes the uncoated region of the implant, can be easily produced and is particularly dimensionally stable. Corners of the cross-section can be rounded.

In one embodiment, one end of the rib-shaped spacers pointing toward the reservoir is designed to be rounded. In this way, insertion of the implant can be simplified and a particularly reproducible coating can be ensured.

In one embodiment, at least one side of the reservoir facing away from the lower part and/or the conical portion is designed as a cylinder. In particular, the apparatus further comprises a piston, corresponding to the cylinder, for pressing the coating agent. The piston is typically a separate component.

The cylinder and piston are shaped accordingly. The piston can typically be moved manually within the cylinder in order to press the coating agent from the reservoir in the direction of the conical portion and/or opening. This is particularly advantageous in the case of viscous or highly viscous bone cements that flow only slowly. In this case, it may happen that the bone cement adhering to the implant is discharged downwards from the conical portion more quickly than bone cement flows from the reservoir. Due to the ability to push the bone cement downwards, defects in the coating due to missing bone cement are prevented and rapid and uniform coating is ensured.

A part of the reservoir or the entire reservoir can be configured as a cylinder. The cylinder does not have to be a circular cylinder but can exhibit any cross-sectional area. However, a circular cylinder is particularly easy to produce. In particular, the piston and the cylinder comprise a common central axis along which the relative movement is carried out. The piston can comprise a U-shaped cross-section. Alternatively, the piston can be designed as a convex hollow body, for example.

In one embodiment, the piston can comprise a central opening through which the implant can be inserted. The opening can be partially or completely closed with a cover, in particular a rubber-elastic cover. Since, as a rule, the plunger is used with highly viscous bone cement, it can push bone cement downwards despite the opening. A wiper can be arranged in the region of the opening.

The reservoir, the lower part and/or the conical portion are made in particular of plastic and/or rubber. Preferably, the reservoir and the lower part, with the exception of the conical portion, are made of plastic and/or the conical portion is made of a rubber-elastic material, such as, e.g., rubber.

A further aspect of the invention is a system, in particular for coating an elongate implant. The system comprises an apparatus according to the invention. The system further comprises an additional lower part having an opening, wherein the material forming the opening is rubber-elastic and serves as a wiper. The additional lower part comprises a second connecting region that corresponds to the first connecting region of the apparatus and can be mechanically reversibly connected thereto.

In this way, the desired layer thickness can be set prior to coating by selecting and connecting a desired conical portion. The system can also be referred to as a kit. In particular, the components of the system are packaged together so that the appropriate conical portion, i.e. the lower part of the apparatus, can be selected and used intraoperatively. In other words, the system comprises an upper part and at least two lower parts. There may be a plurality of additional lower parts, wherein each opening of each lower part exhibits a different shape and/or size.

All features, advantages and embodiments of the apparatus, use and method described above can also apply to the system and vice versa.

In one embodiment, the system further comprises components for producing bone cement. In particular, the components are such that bone cement can be made without the addition of additional materials. For example, the components include polymethyl methacrylate bone cement powder and monomer liquid. In particular, the components are in each case contained in separate packaging. Any active ingredients typically need to be added. In one embodiment, the system further comprises a mixing apparatus for mixing bone cement. The mixing apparatus can comprise a mixing bowl and one or more mixing tools, such as, e.g., one or more spatulas. Alternatively, or additionally, the mixing apparatus can comprise a mixing system, for example with a cartridge having an integrated or mounted mixing rod.

A further aspect of the invention is a use of an apparatus according to the invention for coating an elongate implant. All features, advantages and embodiments of the apparatus, system and method described above can also apply to the use and vice versa.

The implant is in particular a surgical implant, such as, e.g., a locked or non-locked intramedullary nail or an osteosynthesis plate. The implant can be made of, e.g., titanium, titanium alloys, steel, a composite material and/or one or more plastics or can contain one or more of these materials. The implant to be coated can in principle also be any other elongate implant, such as, e.g., an implantable electrode, a tubular vascular prosthesis or a plastic tube.

In principle, other elongate objects can also be coated.

In particular, coating with a pasty coating agent, e.g., bone cement, such as, e.g., polymethyl methacrylate bone cement. Polymethyl methacrylate bone cement is made by mixing polymethyl methacrylate bone cement powder and methyl methacrylate monomer liquid and cures spontaneously within a few minutes through radical polymerization of the methyl methacrylate. In particular, the coating contains at least one pharmaceutical active ingredient suspended or dissolved therein.

Alternatively or additionally, a polymer or a polymer solution can be used. Suitable materials for this purpose include polylactide, polymethyl methacrylate, polyvinyl acetate and/or polyvinyl chloride, which are dissolved in volatile solvents such as acetone and/or chloroform. The volatile solvent can evaporate from the polymer solution after coating and leave an adhesive polymer film on the surface of the implant.

In particular, the coating agent contains at least one pharmaceutical active ingredient, wherein the at least one pharmaceutical active ingredient is preferably an anti-infective agent, such as, e.g., gentamicin, tobramycin, amikacin, vancomycin, dalbavancin, teicoplanin, daptomycin, clindamycin, meropenem, colistin, ampicillin, amoxicillin, ofloxacin, levofloxacin, moxifloxacin, ciprofloxacin, amphotericin B, micafungin, fluconazole or any mixture of at least two of the above. In addition to their actual, usually mechanical function, the coated implants are also suitable for the local release of an active ingredient.

A further aspect of the invention is a method for coating a longitudinal implant using an apparatus. The method comprises providing an apparatus having a reservoir in which a coating agent is held. The method further comprises moving an elongate implant through the coating agent and through an opening in a lower part of the apparatus. Excess coating agent is wiped off from the implant by a wiper on the apparatus.

The movement is carried out in particular in one direction from the reservoir to the conical portion. The movement of the implant through the coating agent can, for example, be carried out in the reservoir and/or in the lower part. The wiping is carried out in particular when leaving the lower part, preferably through an opening that comprises or forms the wiper. The implant can be longer or shorter than the apparatus. An as-yet uncoated part of the implant can be located above the coating agent, while excess coating agent is wiped off from an already coated part of the implant. The implant can pass through the entire apparatus. In particular, the apparatus is an apparatus according to the invention.

In principle, the movement is a relative movement. It is irrelevant whether the implant, the apparatus or both components are moved. For the sake of simplicity, we will simply refer to “moving” or “moving the implant”. All the above-mentioned possibilities of relative movement are always meant.

If the implant comprises a variable cross-section, a thinner side of the implant is coated first. The implant is then moved down into the apparatus with the thinner side first.

In particular, the movement of the implant is initially carried out in such a way that a first end of the implant is located in the region of a second opening, in particular located at the bottom, of the conical portion. The first end can be pushed through the second opening. Subsequently, the coating agent is filled into the reservoir and/or the conical portion. In particular, the apparatus is aligned so that the reservoir is located centrally above the conical portion. If this sequence is followed, it is effectively prevented that coating agent penetrates into an internal cavity of the implant. The background is that intramedullary nails and other implants can be designed as hollow bodies that are open at the ends. In these cases, penetration of coating agent into the cavity is usually not desired.

Once the implant is located in the region of the opening, the material forming the opening can be stretched by contact with the implant.

Due to gravity, the coating agent can sink downwards into the conical portion. The implant can be wetted there. The coating agent then adheres to the surface of the implant.

The implant can be moved downwards relative to the apparatus, for instance by pushing on the upper end and/or pulling on the lower end. Preferably, the entire implant is moved through the apparatus so that each region of the implant comes into contact with the wiper once. Meanwhile, coating agent can flow downwards from the reservoir into the conical portion. This can be assisted by pressing a piston.

Spacers, such as, e.g., rib-shaped spacers, can contact the implant and thus maintain a distance between the wiper and the surface of the implant. In this way, at least in the regions between the spacers, a defined, uniform layer thickness that corresponds to the radial extension of the spacers can be achieved. Excess coating agent is wiped off and remains in the conical portion.

In particular, post-processing of the coating is subsequently carried out for producing the final, solid coating. This can be carried out, for example, by curing the coating agent or by evaporating and/or vaporizing solvent.

In one embodiment, the removal of coating agent is carried out from openings of the implant, for example by plugging in using a pin or plunger. This is preferably carried out prior to post-processing.

### BRIEF DESCRIPTION OF THE DRAWINGS

Exemplary embodiments of the invention are also explained in greater detail below with reference to figures. Features of the exemplary embodiments can be combined individually or in a plurality of the claimed subjects, unless otherwise indicated. The claimed scope of protection is not limited to the exemplary embodiments. In the drawings:

FIG. 1 shows a perspective sectional drawing of an apparatus;

FIG. 2 shows two sectional drawings of an apparatus;

FIG. 3 shows a sectional drawing of an apparatus in use;

FIG. 4 shows a sectional drawing of an apparatus in use;

FIG. 5 shows a coated implant;

FIG. 6 shows an exploded view of an apparatus;

FIG. 7 shows a sectional drawing of an apparatus in use;

FIGS. 8 and 9 show cross-sections of coated implants; and,

FIG. 10 shows a sectional drawing of a spacer.

### DETAILED DESCRIPTION OF THE INVENTION

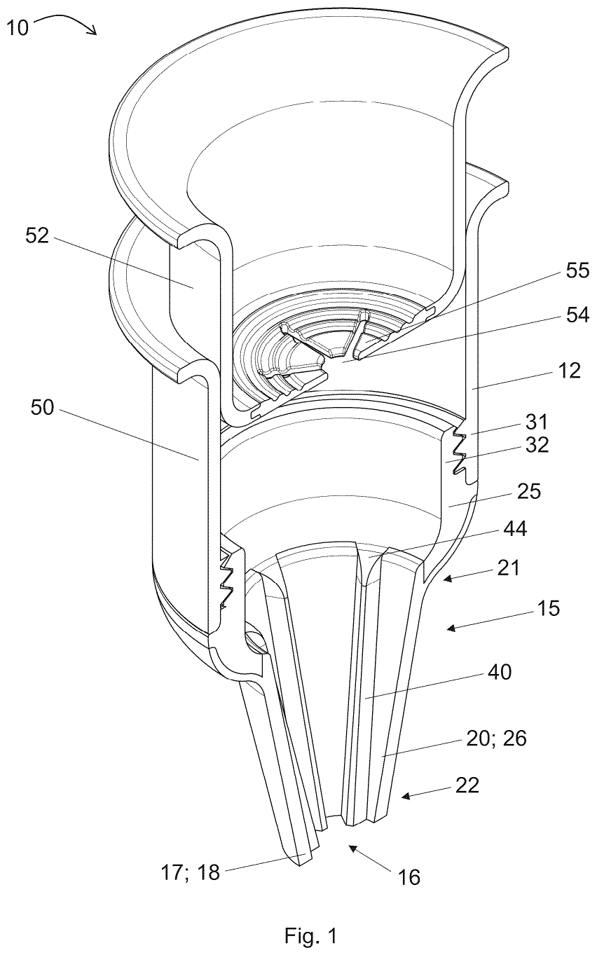

FIG. 1 shows an apparatus 10 according to the invention for coating elongate implants. The apparatus 10 comprises a reservoir 12, shown here in the center, for holding a coating agent. The reservoir 12 has a circular-cylindrical or rotationally symmetrical basic shape and is open at the top side and at the bottom side. On the bottom side, the reservoir 12 comprises a first connecting region 31 in the shape of an internal thread.

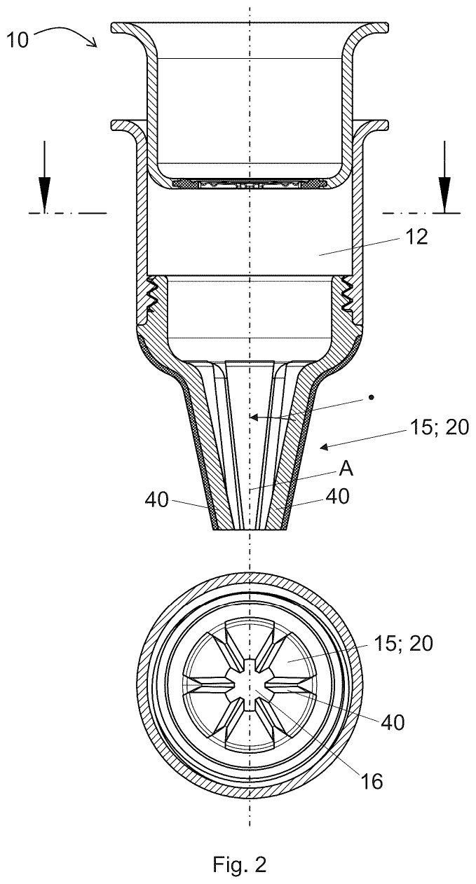

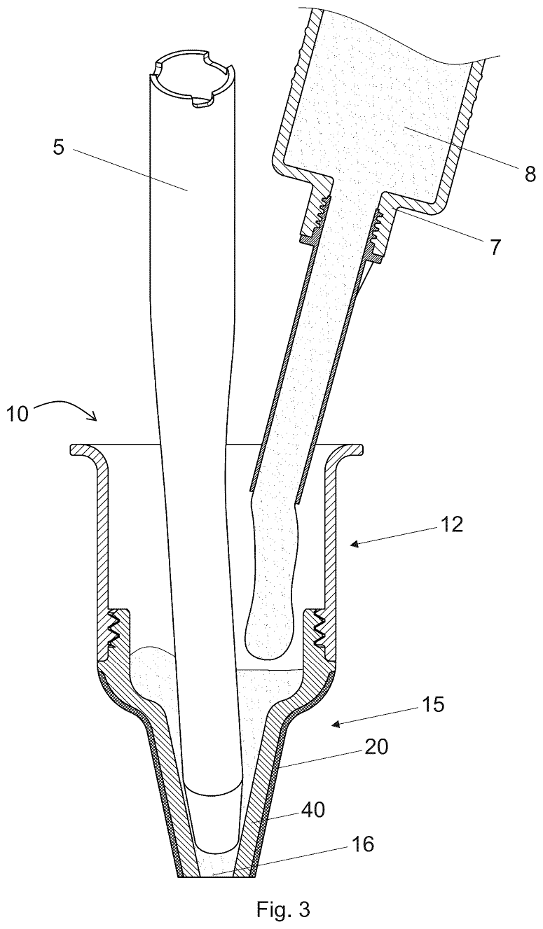

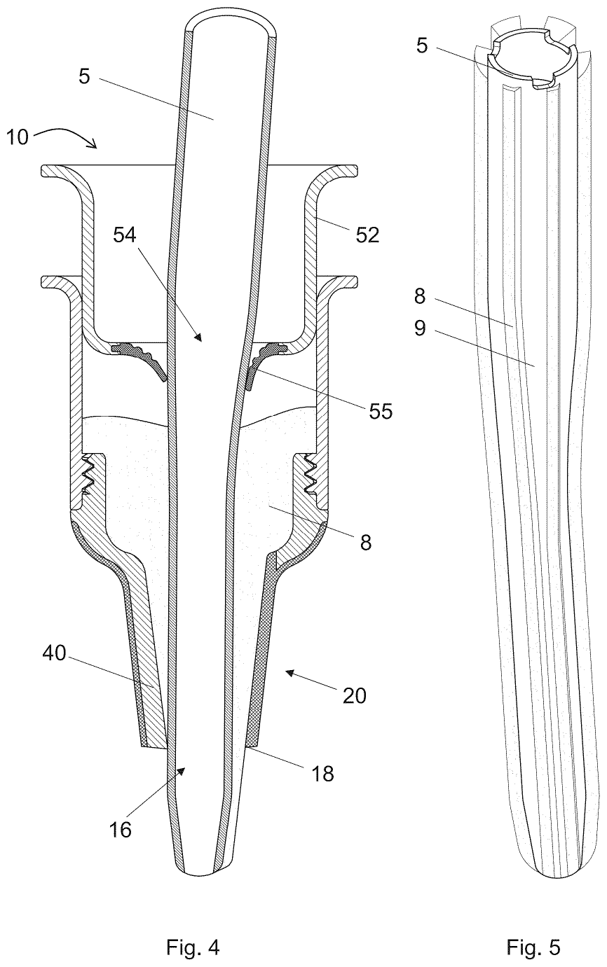

A lower part 15, which here is composed of two components 25, 26 that cannot be separated from one another in a non-destructive manner, is located below the reservoir 12. An approximately annular upper component 25 comprises a second connecting part 32 in the shape of an external thread that can be screwed into the internal thread of the reservoir 12. The upper component 25 can be made of comparatively rigid plastic. A lower component 26 of the lower part 15 is configured as a hollow conical portion 20 that exhibits a basic shape of a truncated cone. The lower component 26 can be made of rubber-clastic material. The larger first side 21 of the conical portion 20 is connected to the upper component 25. The smaller second side 22 of the conical portion 20 points downwards and forms the opening 16 of the apparatus. The opening 16 exhibits a circular basic shape. The exact shape of the opening 16 deviates from the circular shape due to the spacers 40 described below. The material 17 forming the opening 16 is rubber-elastic and serves as a wiper 18 for wiping. In particular, the entire lower component 26 and/or the entire conical portion 20 is rubber-clastic. The use of the apparatus 10 is shown in FIGS. 3 and 4. The upper component 25 can comprise the spacers 40 described below, as shown in FIG. 2.

In the lower part 15, namely in the conical portion 20, spacers 40 are arranged in order to ensure a defined distance between the surface of the implant and the wiper 18. The spacers 40 are configured as ribs that are substantially vertical or aligned along the central axis A or longitudinal axis of the apparatus 10 and/or the conical portion 20. This prevents the spacers 40 from yielding in the vertical direction, which is also referred to as the z-direction. The upper end 44 of the spacers pointing toward the reservoir 12 is designed to be rounded. In this way, the unwanted blocking of the implant during downward movement of the implant can be prevented.

The embodiment shown here shows three spacers 40 in one half of the apparatus 10, i.e. a total of six spacers 40. The spacers 40 are uniformly distributed in the circumferential direction.

The apparatus optionally further comprises a piston 52. In this case, the upper side of the reservoir 12 is cylindrical, here circular-cylindrical. The piston 52 fits exactly into the upper side of the reservoir 12 and serves to press a highly viscous coating agent downwards into the conical portion 20. The piston 52 comprises an opening 54 that is at least partially closed by a rubber-elastic cover 55, in particular one having star-shaped slits.

FIG. 2 shows a longitudinal section of an apparatus 10 at the top and a cross-section at the bottom, with the viewing direction indicated in the longitudinal section. The apparatus 10 can correspond entirely or partially to the apparatus 10 of FIG. 1. The cut extends in the region of the conical portion 20 on both sides through respective spacers 40. The angle α between a longitudinal axis of a rib-shaped spacer 40 and a central axis A of the apparatus 10, which extends vertically here, corresponds to the angle of the inner surface of the conical portion 20 to the central axis A of the apparatus.

In the lower representation, it can be seen that the opening 16, which exhibits a circular basic shape, is star-shaped due to the spacers 40.

FIG. 3 shows the use of the apparatus 10. An implant 5, in particular an intramedullary nail, is arranged approximately vertically in the apparatus. A coating agent 8, such as, e.g., bone cement containing an active ingredient, is introduced from above into the reservoir 12 and/or the lower part 15, for example using a syringe 7. At this time, the lower end of the implant 5 already rests against the spacers 40 and/or the inner surface of the conical portion 20 or has already been pushed a little way through the opening 16. In this way, coating agent 8 is prevented from entering the interior of the hollow implant 5.

FIG. 4 shows a further step in the use of the apparatus 10. In this representation, the section extends in the region of the conical portion 20 on the left side through a spacer 40 and on the right side through a region between two spacers, in which no spacer is present. The implant 5 has been moved further downwards, approximately through the middle of the apparatus. The wall of the opening 16 on the right side acts as a wiper 18 and wipes off and retains excess coating agent 8, thereby establishing a constant layer thickness of the coating agent 8 on the implant 5. On the right side, however, the spacer 40 contacts the implant 5 and thus prevents local deposition of coating agent 8.

It is also shown that the implant exhibits a diameter that increases toward the top. Due to the rubber-clastic properties of the conical portion 20, the opening 16 can widen, so that a constant layer thickness can be achieved in this case as well.

In addition, as also shown in FIG. 4, a piston 52 can be used in order to press the coating agent 8 downwards. It is shown how the implant 5 is passed through the opening 54 of the piston 52. The flexible cover 55 of the opening 54 is deflected.

Coated implant 5 is shown by way of example in FIG. 5. The implant comprises strips of coating agent 8 extending along its longitudinal axis, which are spaced apart from one another by gaps 9 therebetween. The gaps correspond to the positions in which the spacer contacted the implant 5.

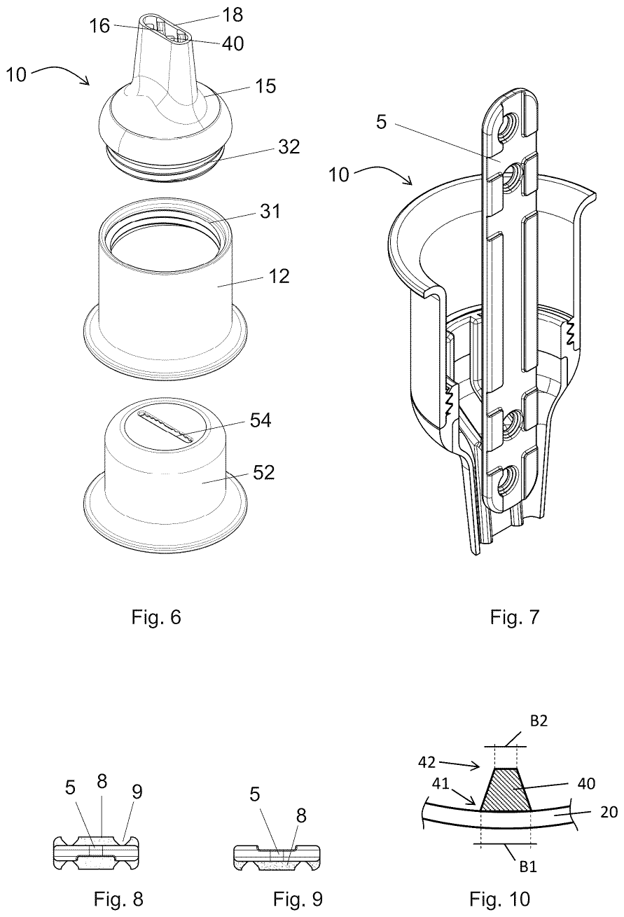

A further embodiment of an apparatus is shown in FIG. 6 using an inverted exploded view. The lower part 15 shown above comprises a slit-shaped opening 16, in which in each case two rib-shaped spacers 40 are visible on the two long sides. The conical portion is also slit-shaped or has an elongate cross-section. This apparatus serves, as shown by way of example in FIG. 7 using an osteosynthesis plate, to coat flat implants 5. The opening 54 in the optional piston 52 can then also be designed in a slit shape. In principle, the basic shape of the opening is adapted to the outer cross-section of the implant and can be selected arbitrarily.

FIGS. 8 and 9 show coated implants 5 in cross-section. Osteosynthesis plates are shown for flat implants 5 by way of example. Typically, only the long sides seen in cross-section are coated. If the spacers are arranged on both sides as in FIG. 6, the pattern shown in FIG. 8 is created with coating agent 8 arranged on both sides and gaps 9 located in between. If, however, spacers are arranged only on one side, the pattern shown in FIG. 9 is created. Here, coating agent 8 is arranged only on one side, having gaps 9 located in between in each case, and on the other side the entire coating agent has been wiped off and retained by the wiper.

FIG. 10 shows a cross-section through a rib-shaped spacer 40, with the viewing direction along an inner surface of the conical portion 20. The spacer here exhibits a trapezoidal shape by way of example. The contact side 41 of the spacer 40 rests against the inner surface of the conical portion 20 and the free side 42 projects freely radially inward for contacting the implant. The width B2 of the free side 42 is less than the width B1 of the contact side 41 measured parallel thereto.

### REFERENCE NUMERALS

5 Implant
7 Syringe
8 Coating agent
9 Gap
10 Apparatus
12 Reservoir
15 Lower part
16 Opening
17 Material
18 Wiper
20 Conical portion
21 First side
22 Second side
25 Upper component
26 Lower component
31 First connecting region
32 Second connecting region
40 Spacer
41 Contact side
42 Free side
44 End
B1 First width (BS)
B2 Second width (FS)
50 Cylinder
52 Piston
54 Opening
55 Cover
A Central Axis
α Angle

1. An apparatus for coating an elongate implant, comprising a reservoir for holding a coating agent and a lower part connected or connectable to the reservoir and having an opening, wherein a material forming the opening is rubber-elastic and serves as a wiper for wiping off excess coating agent.

2. The apparatus according to the claim 1, wherein the lower part comprises a hollow conical portion, wherein the conical portion is made in particular of the rubber-elastic material.

3. The apparatus according to claim 1, wherein a larger first side of the conical portion points toward the reservoir and a smaller second side of the conical portion points away from the reservoir, wherein the opening is located on the second side.

4. The apparatus according to claim 2, wherein the reservoir comprises a first connecting region and the lower part comprises a corresponding second connecting region, and the first connecting region and the second connecting region are mechanically reversibly connectable to one another.

5. The apparatus according to claim 1, wherein the opening exhibits a circular or slit-shaped basic shape.

6. The apparatus according to claim 1, wherein spacers are arranged on the wiper in order to ensure a distance between the wiper and an elongate implant to be coated.

7. The apparatus according to claim 1, wherein the spacers are arranged in a manner uniformly distributed in the circumferential direction.

8. The apparatus according to claim 6, wherein the spacers are configured to be rib-shaped and extend at an angle of less than 30° to a central axis of the apparatus.

9. The apparatus according to claim 1, wherein the rib-shaped spacers comprise a cross-section having a contact side and a free side, wherein a first width of the spacers on the contact side is at least in some regions larger than a second width of the spacers on the free side, measured parallel to the first width.

10. The apparatus according to claim 8, wherein the rib-shaped spacers comprise a triangular, trapezoidal, rectangular or hemispherical cross-section.

11. The apparatus according to claim 8, wherein one end of the rib-shaped spacers pointing toward the reservoir is designed to be rounded.

12. The apparatus according to claim 1, wherein at least one side of the reservoir facing away from the lower part is designed as a cylinder, wherein the apparatus further comprises a piston, corresponding to the cylinder, for pressing the coating agent.

13. The apparatus according to claim 6, wherein the lower part comprises a hollow conical portion into which the coating agent can sink by gravity during use of the apparatus.

14. A system comprising an apparatus according to claim 4 and an additional lower part having an opening, wherein the material forming the opening is rubber-elastic and serves as a wiper, wherein the additional lower part comprises a second connecting region that corresponds to the first connecting region of the apparatus and can be mechanically reversibly connected thereto.

15. A use of an apparatus according to claim 1 for coating an elongate implant.

16. A method for coating an elongate implant using an apparatus, comprising providing an apparatus having a reservoir in which a coating agent is held, moving an elongate implant through the coating agent and through an opening in a lower part of the apparatus, wherein excess coating agent is wiped off the implant by a wiper of the apparatus.

17. The apparatus according to claim 3, wherein the reservoir comprises a first connecting region and the lower part comprises a corresponding second connecting region, and the first connecting region and the second connecting region are mechanically reversibly connectable to one another.

18. The apparatus according to claim 9, wherein one end of the rib-shaped spacers pointing toward the reservoir is designed to be rounded.

19. The apparatus according to claim 10, wherein one end of the rib-shaped spacers pointing toward the reservoir is designed to be rounded.

20. The apparatus according to claim 9, wherein the rib-shaped spacers comprise a triangular, trapezoidal, rectangular or hemispherical cross-section.
